(19) United States
(12) Patent Application Publication
(10) Pub. No.: US 2021/0183477 A1
(43) Pub. Date: Jun. 17, 2021

(54) RELIEVING CHRONIC SYMPTOMS THROUGH TREATMENTS IN A VIRTUAL ENVIRONMENT

(71) Applicant: VR Health Ltd., Tel Aviv (IL)

(72) Inventors: Naomi Rudich, Tel Mond (IL); Dan Lavi, Ariel (IL); Limor Shilony-Nalaboff, Hod Hasharon (IL)

(21) Appl. No.: 17/188,738

(22) Filed: Mar. 1, 2021

Related U.S. Application Data
(63) Continuation of application No. PCT/IB19/00981, filed on Aug. 28, 2019.
(60) Provisional application No. 62/723,632, filed on Aug. 28, 2018.

(51) Int. Cl. G16H 10/20; H04L 29/06; H04L 29/08; G06F 3/0482; G16H 70/20; G16H 10/60; G16H 50/20; G16H 20/70

(52) U.S. Cl. CPC G16H 10/20; H04L 67/38; H04L 67/125; G06F 3/0482; G16H 20/70; G16H 70/20; G16H 10/60; G16H 50/20; H04L 67/36

(57) ABSTRACT

The various embodiments disclosed herein includes a virtual environment (VE) system, and method thereof for reducing chronic symptoms in relation to menopause and chronic diseases such as, but not limited to, cancer. The symptoms include, but are not limited to, hot flashes, anxiety, chemo brain, immune system, stress, pain, fatigue, hair loss, nerve and muscle problems such as numbness and tingling, mood changes, and so on.

### BACKGROUND

Chronic diseases, such as cancer, affect millions of Americans representing over 40% of the population. To treat many of the symptoms, treatments may be done which come along with side effects, all of which can cause other symptoms such as anxiety and depression.

One of the most common chronic symptoms that women suffer from are hot flashes. A Hot flash symptom is a chronic symptom of menopause in which that affects nearly 80% of all women. Hot flashes may also be caused by a variety of other diseases, most notably breast cancer and thyroid problems such as hyperthyroidism. A hot flash is a sudden, intense, hot feeling in the face and upper body that can also be accompanied by anxiety, dizziness, sweat and the like.

Existing solutions for relieving the effects of hot flashes include drugs such as Venlafaxine and Gabapentin that remedy some of the symptoms. However, patients are often discouraged to take such drugs due to the many other potential side effects. Furthermore, the efficiency of such drugs is not always proven to be found as helpful for hot flashes and anxiety. Furthermore, woman suffering from hormonal affected cancer are prohibited from using such drugs.

A social worker, nurse, personal coach, and psychologist are trained to evaluate the stress of the patient and conduct some exercise to reduce the stress, and thus the anxiety of the patient. However, personal sessions are pre-scheduled and typically cannot be provided on-demand. Thus, the symptoms and side effects cannot be relieved as the patient experiences them. Other solutions include applications (apps) that play relaxing music, provide interactive games or offer general psychological support. Such applications are not customized to the patient's individual needs and thus cannot provide an ideal treatment.

It would therefore be advantageous to provide a solution that would overcome the deficiencies of the prior art.

### BRIEF SUMMARY

Systems, methods, and computer program products of the present invention providing a virtual environment to the user via a virtual or augmented reality system. A method of the present disclosure includes presenting a questionnaire to a user via a virtual or augmented reality system. User input is received in response to the questionnaire. A virtual environment is determined based on the user input. The virtual environment is provided to the user via the virtual or augmented reality system. A plurality of biometric measurements are determined by a plurality of sensors. Whether at least one of the plurality of biometric measurements is above a predetermined baseline is determined. When the at least one of the plurality of biometric measurements is above the predetermined baseline, the virtual environment is modified based on the at least one of the plurality of biometric measurements.

A system of the present disclosure includes a virtual or augmented reality display adapted to display a virtual environment to a user, a plurality of sensors coupled to the user, and a computing node including a computer readable storage medium having program instructions embodied therewith. The program instructions are executable by a processor of the computing node to cause the processor to perform a method including presenting a questionnaire to a user via the virtual or augmented reality display. User input is received in response to the questionnaire. A virtual environment is determined based on the user input. The virtual environment is provided to the user via the virtual or augmented reality system. A plurality of biometric measurements are determined by the plurality of sensors. Whether at least one of the plurality of biometric measurements is above a predetermined baseline is determined. When the at least one of the plurality of biometric measurements is above the predetermined baseline, the virtual environment is modified based on the at least one of the plurality of biometric measurements.

A computer program product relieving chronic conditions in a user of the present disclosure includes a computer readable storage medium having program instructions embodied therewith. The program instructions are executable by a processor to cause the processor to perform a method including presenting a questionnaire to a user via a virtual or augmented reality system. User input is received in response to the questionnaire. A virtual environment is determined based on the user input. The virtual environment is provided to the user via the virtual or augmented reality system. A plurality of biometric measurements are determined by a plurality of sensors. Whether at least one of the plurality of biometric measurements is above a predetermined baseline is determined. When the at least one of the plurality of biometric measurements is above the predetermined baseline, the virtual environment is modified based on the at least one of the plurality of biometric measurements.

### BRIEF DESCRIPTION OF THE DRAWINGS

The foregoing and other objects, features, and advantages of the disclosed embodiments will be apparent from the following detailed description taken in conjunction with the accompanying drawings.

FIGS. 1A-1B illustrate various network diagrams according to embodiments of the present disclosure.

FIG. 2 is a flowchart illustrating a method for performing a therapy session to relieve chronic symptoms according to embodiments of the present disclosure.

FIG. 3 is a flowchart illustrating another method for performing a therapy session to relieve chronic symptoms according to embodiments of the present disclosure.

FIG. 4 is a screenshot of an initial virtual environment (VE) demonstrating a snowy day of part of a wintery scenery according to embodiments of the present disclosure.

FIG. 5 is a screenshot of a modified VE demonstrating a severely snowy day of part of a wintery scenery according to embodiments of the present disclosure.

FIG. 6 illustrates an exemplary virtual reality headset according to embodiments of the present disclosure.

FIG. 7 is a flow chart illustrating an exemplary method for relieving chronic symptoms through treatments in a virtual environment according to embodiments of the present disclosure.

FIG. 8 depicts an exemplary computing node according to embodiments of the present disclosure.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various embodiments disclosed herein includes a virtual environment (VE) system, and method thereof for reducing chronic symptoms in relation to menopause and chronic diseases such as, but not limited to, cancer. The symptoms include, but are not limited to, hot flashes, anxiety, chemo brain, immune system, stress, pain, fatigue, hair loss, nerve and muscle problems such as numbness and tingling, mood changes, and so on.

In various embodiments, a combination of active feedback from the user and passive feedback from but not limited to sensors placed on a head mounted device (HMD) or individual sensors placed on the body and head, provide the data necessary for the artificial intelligence (AI) system to psychologically assist the user by displaying a VE in the HMD in order to calm the user and reduce the severity of the hot flashes or other symptoms.

In an embodiment, the user is asked a series of questions by a virtual coach in order to accurately determine the medical and psychosocial condition of the user. Further, sensory signals may be collected from one or more biofeedback sensors attached to the user or on the HMD. Based on at least one of the answers to these questions and the sensory data, a VE (in which you go through a specific and personalized therapy session) is presented on the HMD. Such an environment immerses the user in a psychological and therapeutic session. This session helps engage the brain in controlling the physical symptoms and psychological distress during the healing process.

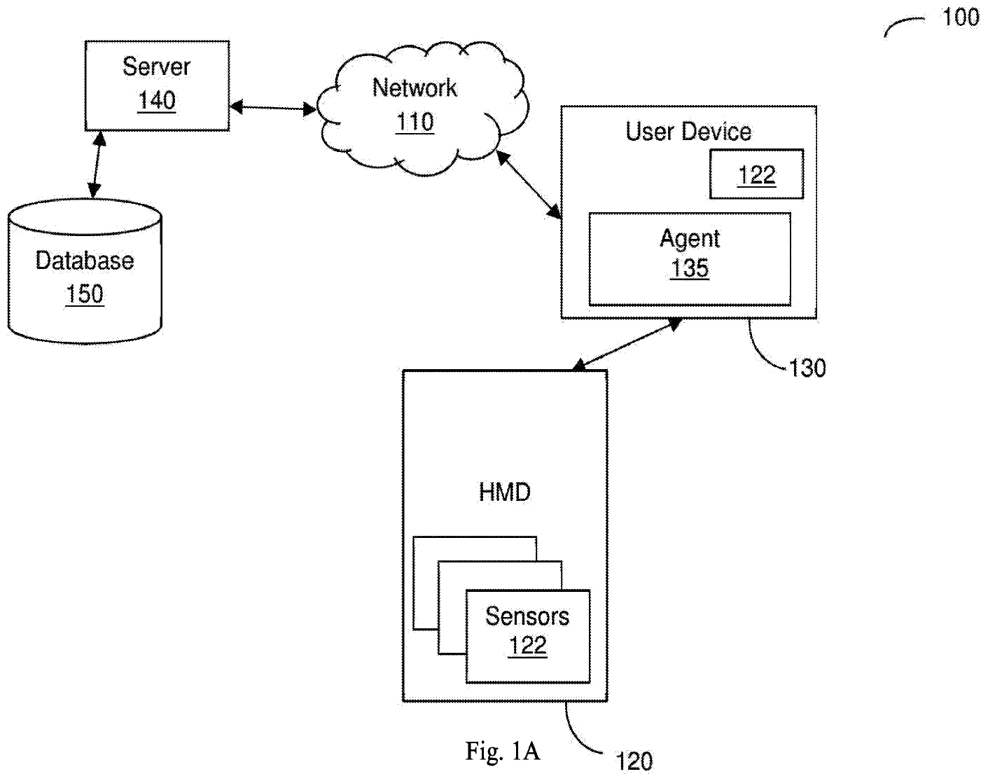

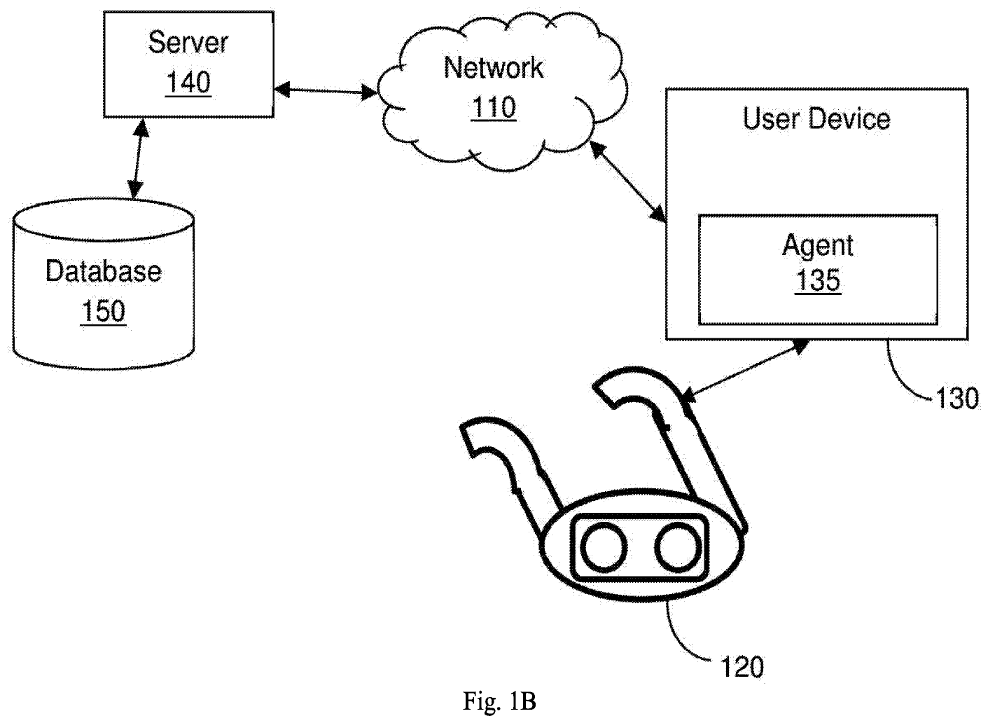

FIG. 1A shows a diagram utilized to describe the operation of the virtual experience (VE) system 100 according to various disclosed embodiments. The VE system 100 includes a head mounted device (HMD) 120 connected to a user device 130. In some configurations, the VE system 100 also includes a remote server 140 connected to a database 150. The user device 130 may be communicatively connected to a network 110 which further may be remotely controlled by the remote server 140. In an embodiment, the user device may be communicatively connected via a network 110. The network 110 may be, but is not limited to, a wireless, cellular or wired network, a local area network (LAN), a wide area network (WAN), a metro area network (MAN), the Internet, the worldwide web (WWW), similar networks, or any combination thereof.

The VE system 100 may further include one or more biofeedback sensors (collectively shown as sensors 122). The sensors 122 may include, but are not limited to, heart rate variability sensors, body temperature sensors, an oxygen meter to detect the user's blood oxygen level, eye tracking sensors, position sensors for tracking the head's position, direction, and motion of the head of the user, blood pressure sensors, stress sensors, Electromyography (EMG) sensors, Galvanic Skin Response (GSR) sensors, Electrocardiography (EKG) sensors, Voice detection, and so on. The biofeedback sensors 122 may be in a form of a wearable device, electrodes attached to the skin, and other configurations capable of monitoring physical processes and functions of the user. Some of the sensors 122 may be included in the user device 130 and/or the HMD 120.

The signals from the sensors 122 mounted in the HMD 120 may be transmitted to the user device 130 over a cable, e.g., a USB cable, or over a wireless medium using protocols, such as Bluetooth, Wi-Fi, BLE, ZigBee, and the like. The sensory signals may also be transmitted to the server 140 as data packets over the network 110. In any configuration, the user can answer questions inside the VE experience by gazing at the answer on the screen, by voice, or by touching it with using HMD's 120 touch element.

According to the disclosed embodiments, signals collected from the sensors 122 may be utilized to, for example, determine parameters associated with a current state of the user. Such parameters may be further utilized to determine when the user is prone to experiencing hot flashes, increased anxiety levels, etc. and what kind of simulations reduce those experiences.

The user device 130 may be connected to the HMD 120 via a cable (e.g., HDMI cable or micro USB) or over a wireless connection. The wireless connection may include a Bluetooth, a Wi-Fi, a Wi-Gig, and the like. In some configurations, the user device 130 acts as the headset's display and processor, while the HMD 120 itself acts as the controller for controlling the field of view and the rotational tracking. For example, the HMD 120 may be designed to allow for a smart phone to be inserted behind the lens of the HMD 120. In such configurations, the HDM 120 may include audio means.

In another configuration, the HMD 120 is conventionally structured to include a small display. For example, the HMD 120 may comprise a housing having a liquid-crystal display (LCD) for displaying images, an optical means (lenses) for guiding the images projected on this LCD toward both eyes of a user, and auditory speakers that are aligned with a user's ears to play music or other sound recordings. Visual images and accompanying audio of a virtual environment can be transmitted from the server 140 or the user device 130 to the HMD 120, such that the images are displayed via the small display and the accompanying audio is played through the speakers.

The user device 130 may be, but is not limited to, a personal computer, a laptop, a tablet computer, a smartphone, a wearable computing device, or any other device capable of receiving, storing, sending, and displaying data. In an embodiment, a user device 130 may be installed with an agent 135 which may be, but is not limited to, a software application. An application executed or accessed through the user device 130 may be, but is not limited to, a mobile application, a virtual application, a web application, a native application, and the like. In an embodiment, the agent 135 may be configured to receive information on instructions from the remote server 140 in response to inputs (such as sensory signals and user's interactions) provided by the user device 130. Alternatively, the agent 135 may be configured to operate in an off-line mode, i.e., without an active connection to the network 110 or the server 140.

It should be appreciated that the agent 135 is stored on in a machine-readable media (not shown) in the user device 130. Software executed by the agent 135 shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing system to perform the various functions described herein

According to the disclosed embodiments, the agent 135 is executed when a user wishes to start a therapy session. Then, the agent 135 selects an experience to be trained using the therapy session. The session may be one session being part of a psychological treatment protocol. The protocol may be one of many psychological treatment protocols, each selected to address different medical conditions, symptoms and/or side effect. A psychological treatment protocol may define a number of sessions required, a goal for each session, and/or the experience to be trained.

In a non-limiting embodiment, the experience may be rendered based on a goal set for the therapy and a set of personal and/or environmental parameters. Personal parameters may include age, general medical conditions, personal preferences, and so on. General medical conditions may include a cancer type, stage of cancer (e.g., breast cancer), type of therapy (chemotherapy, lumpectomy, mastectomy, etc.), surgical history, drugs being taken, and so on. The personal parameters may be retrieved from the database 150 or stored in the agent 135. I n such embodiments, when some or all of the personal parameters are not available, the user is prompted to enter the missing information. Alternatively, the user may be prompted to confirm or update the accuracy of such parameters.

The environmental parameters may include a current location (e.g., home, clinic, or hospital), current weather, current time, and so on. It should be noted the experience may be rendered in response to personal parameters, environmental parameters, or both. Alternatively, the experience may be a default experience not based on any of these parameters. In an embodiment, a selection of the experience would render a background image on the HMD's display.

Then, a series of questions attempting to determine the current physical conditions of the user are presented on the HMD's display. Such questions may be related to, for example, a current mode of feeling (e.g., What feeling are you experiencing right now?); a level of intensity and frequency of the symptoms or side effects (e.g., What is the level of your hot flash?); and how the user experienced the symptoms (e.g., Are your hot flashes making you frustrated? agitated? tired?). The questions may also be related to or based on what has been done from the last session to remedy the symptoms (e.g., Has resisting your hot flashes made it easier to deal with them?).

In an embodiment, the answers to the questions are multiple-choice options or open answers where the user can gaze to the answer or answer by voice or, in some embodiments, by touch, best describing her current conditions. The answers to the questions are captured and processed by the agent 135. In response, the agent 135 may modify the initial environment to better match the current conditions of the user. For example, if the current physical conditions demonstrate a higher level and frequency of hot flashes than the current sessions, then the initial environment may be modified. An example VE environment is discussed below. It should be noted that the agent 135 may provide a set of questions in response to a particular answer. For example, a first question may be:

“Have you experienced frequent hot flashes?”

If the answer is “yes”, a subsequent question may be:

“Please select an approximate frequency: a. every day; b. every week; c. every month”

In an embodiment, the agent 135 implements an AI engine to analyze the answers and renders the VE that would best serve the current physical and psychological conditions. The rendered environment is displayed to the user. While the user interacts with the VE, sensory signals from one or more sensors 120 may be collected. In an embodiment, at least one of the previously mentioned sensors is utilized.

In some embodiments, the AI engine is a learning system. In various embodiments, a feature vector is provided to the learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Artificial neural networks (ANNs) are distributed computing systems, which consist of a number of neurons interconnected through connection points called synapses. Each synapse encodes the strength of the connection between the output of one neuron and the input of another. The output of each neuron is determined by the aggregate input received from other neurons that are connected to it. Thus, the output of a given neuron is based on the outputs of connected neurons from preceding layers and the strength of the connections as determined by the synaptic weights. An ANN is trained to solve a specific problem (e.g., pattern recognition) by adjusting the weights of the synapses such that a particular class of inputs produce a desired output.

The sensory signals collected from the biofeedback sensors are compared to a baseline or a plurality of baselines. The baseline determines a normal expected response to a particular VE. The baseline can be determined for each user based in part on the personal parameters and/or information learnt during previous sessions for the user or group of users having similar personal parameters. In an embodiment, the baselines can be adjusted based, in part, on the environmental parameters. For example, if the therapy session is performed when the user is in the hospital, a HRV baseline would be higher relative to a therapy session performed at a relaxing home environment.

The baseline can be determined based on statistical techniques, such as average, moving average, Grubbs, and the like. Deviations from the baselines can be detected using frequencies analysis, Hidden Markov Models, Kolmogorov-Smirnov, U-Test and the like.

In another embodiment, the collected sensory signals can be fed to a machine learning model trained to determine if the VE should be adjusted. That is, the model is trained to classify sensory signals collected from one or more sensors to the appropriate VE environment.

The VE created in FIG. 1A may include, but is not limited to, virtual reality (VR), augmented reality (AR), mixed reality (MR), games, video, and the like.

It should be noted that modifying the VE and psychological session content (texts) may include modifying any or a combination of following features: sound, icons, avatars, colors, and background. The specific features of the VE to be modified is determined by the agent 135 and/or the server 140 in response to analysis of the sensory signal.

In an embodiment, the VE may be rendered to include a real-time feedback to the user. Such a feedback is generated based on the collected sensory signals. For example, a thermometer can be displayed providing indication on the measured body temperature.

It should be noted that sensory signals are collected and analyzed during the entire session and the VE adaptive changes respective thereof. At the end of the session, the success of treatment is determined based on the readings of the biofeedback sensors and/or a feedback provided by the user. The treatment evaluation may be utilized to determine whether a level of exercises was efficient or not. In an embodiment, this information can be saved for future analysis.

FIG. 1B shows another diagram utilized to describe the operation of the virtual environment (VE) system 100 according to various disclosed embodiments. The VE system 100 includes a head mounted device (HMD) 120 connected to a user device 130. In some configurations, the VE system 100 also includes a remote server 140 connected to a database 150.

In this alternative embodiment, the HMD 120 provides a platform for the AI in the user device 130 to communicate with the user and provide the environment necessary to treat the patient. The VE created in FIG. 1B may include, but is not limited to, virtual environment (VR), augmented reality (AR), mixed reality (MR), games, video, and the like.

The user device 130 may be communicatively connected to a network 110 which further may be remotely controlled by the remote server 140. In an embodiment, the user device may be communicatively connected via a network 110. The network 110 may be, but is not limited to, a wireless, cellular or wired network, a local area network (LAN), a wide area network (WAN), a metro area network (MAN), the Internet, the worldwide web (WWW), similar networks, or any combination thereof.

The user device 130 may be connected to the HMD 120 via a cable (e.g., HDMI cable or micro USB) or over a wireless connection. The wireless connection may include a Bluetooth, a Wi-Fi, a Wi-Gig, and the like. In some configurations, the user device 130 acts as the headset's display and processor, while the HMD 120 itself acts as the controller for controlling the field of view and the rotational tracking. For example, the HMD 120 may be designed to allow for a smart phone to be inserted behind the lens of the HMD 120. In such configurations, the HDM 120 may include audio means.

In another configuration, the HMD 120 is conventionally structured to include a small display. For example, the HMD 120 may comprise a housing having a LCD for displaying images, an optical means (lenses) for guiding the images projected on this LCD toward both eyes of a user, and auditory speakers that are aligned with a user's ears to play music or other sound recordings. Visual images and accompanying audio of a virtual environment can be transmitted from the server 140 or the user device 130 to the HMD 120, such that the images are displayed via the small display and the accompanying audio is played through the speakers.

The user device 130 may be, but is not limited to, a personal computer, a laptop, a tablet computer, a smartphone, a wearable computing device, or any other device capable of receiving, storing, sending, and displaying data. In an embodiment, a user device 130 may be installed with an agent 135 which may be, but is not limited to, a software application. An application executed or accessed through the user device 130 may be, but is not limited to, a mobile application, a virtual application, a web application, a native application, and the like. In an embodiment, the agent 135 may be configured to receive information on instructions from the remote server 140 in response to inputs provided by the user device 130. Alternatively, the agent 135 may be configured to operate in an off-line mode, i.e., without an active connection to the network 110 or the server 140.

It should be appreciated that the agent 135 is stored on in a machine-readable media (not shown) in the user device 130. Software executed by the agent 135 shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing system to perform the various functions described herein

According to the disclosed embodiments, the agent 135 is executed when a user wishes to start a therapy session. Then, the agent 135 selects an experience to be trained using the therapy session. The session may be one session being part of a psychological treatment protocol. The protocol may be one of many psychological treatment protocols, each selected to address different medical conditions, symptoms and/or side effect. A psychological treatment protocol may define a number of sessions required, a goal for each session, and/or the experience to be trained.

In a non-limiting embodiment, the experience may be rendered based on a goal set for the therapy and a set of personal and/or environmental parameters. Personal parameters may include age, general medical conditions, personal preferences, and so on. General medical conditions may include a cancer type, stage of the cancer (e.g., breast cancer), type of therapy (chemotherapy, lumpectomy, mastectomy, etc.), surgical history, drugs being taken, and so on. The personal parameters may be retrieved from the database 150 or stored in the agent 135. In such embodiments, when some or all of the personal parameters are not available, the user is prompted to enter the missing information. Alternatively, the user may be prompted to confirm or update the accuracy of such parameters.

The environmental parameters may include a current location (e.g., home, clinic, or hospital), current weather, current time, and so on. It should be noted the experience may be rendered in response to personal parameters, environmental parameters, or both. Alternatively, the experience may be a default experience not based on any of these parameters. In an embodiment, a selection of the experience would render a background image on the HMD's display.

Then, a series of questions attempting to determine the current physical conditions of the user are presented on the HMD's display. Such questions may be related to, for example, a current mode of feeling (e.g., What feeling are you experiencing right now?); a level of intensity and frequency of the symptoms (e.g., What is the level of your hot flash?); and how the user experienced the symptoms (e.g., Are your hot flashes making you frustrated? agitated? tired?). The questions may also be related to or based on what has been done from the last session to remedy the symptoms (e.g., Has resisting your hot flashes made it easier to deal with them?).

In an embodiment, the answers to the questions are multiple-choice options or open answers where the user can gaze to the answer or answer by voice or, in some embodiments, by touch, best describing her current conditions. The answers to the questions are captured and processed by the agent 135. In response, the agent 135 may modify the initial environment to better match the current conditions of the user. For example, if the current physical conditions demonstrate a higher level and frequency of hot flashes than the current sessions, then the initial environment may be modified. An example VE environment is discussed below. It should be noted that the agent 135 may provide a set of questions in response to a particular answer. For example, a first question may be:

“Have you experienced frequent hot flashes?”

If the answer is “yes”, a subsequent question may be:

“Please select an approximate frequency: a. every day; b. every week; c. every month”

In an embodiment, the agent 135 implements an AI engine to analyze the answers and renders the VE that would best serve the current physical conditions and psychological conditions. The rendered environment is displayed to the user.

In another embodiment, using an immersive VE the user is taught to pace breathe through visual and audio cues during the coaching session in order to help reduce the hot flashes. The session requires the user to breathe 6 breaths per minute from the diaphragm. For example, the user can be coached to inhale 5 times and exhale 5 times in a coaching session lasting typically between 3 to 15 minutes.

It should be noted that modifying the VE environment and psychological session content (texts) may include modifying any or a combination of the following features: sound, icons, avatars, colors, and background. The specific features of the VE environment to be modified is determined by the agent 135 and/or the server 140.

In an embodiment, the VE may be rendered to include a real-time feedback to the user.

At the end of the session, the success of treatment is determined based on the feedback provided by the user and to what extent has the user's symptoms have subsided. The treatment evaluation may be utilized to determine whether a level of exercises was efficient or not. In an embodiment, this information can be saved for future analysis.

An example for selecting an experience and modifying the VE environment is provided below.

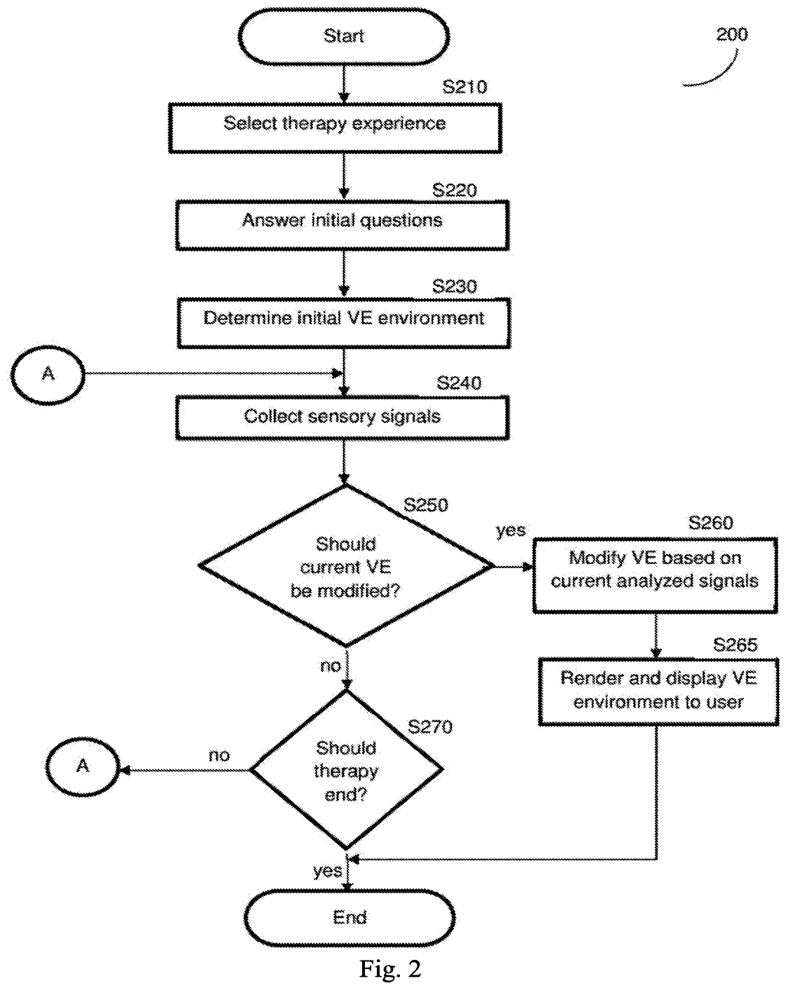

It should be noted that the method of FIG. 2 has been described with reference to an embodiment related to breast cancer treatments. However, the disclosed embodiments can be utilized for relieving symptoms and side effects related to chronic diseases, such as obesity, depression, and the like. Furthermore, women suffering from hot flashes during menopause can be treated using the VE system and methods disclosed herein.

It should be noted that any of the user device 130 and remote server 140 includes at least a processing circuitry coupled to a memory. The processing circuitry can be accomplished through one or more processors that may be implemented with any combination of general-purpose microprocessors, multi-core processors, microcontrollers, digital signal processors (DSPs), field programmable gate array (FPGAs), programmable logic devices (PLDs), controllers, state machines, gated logic, discrete hardware components, dedicated hardware finite state machines, or any other suitable entities that can perform calculations or other manipulations of information.

The memory may include any type of machine-readable media for storing software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing circuitry to perform the various functions described herein.

It should be understood that the embodiments disclosed herein are not limited to the specific architecture illustrated in FIGS. 1A-1B, and other architectures may be equally used without departing from the scope of the disclosed embodiments. Specifically, the remote server 140 may reside in a cloud computing platform, a datacenter, and the like. Moreover, in an embodiment, there may be a plurality of user devices operating as described hereinabove. It should further be noted that the components of the network diagram 100 may be geographically distributed without departing from the scope of the disclosure.

FIG. 2 is an example flowchart 200 illustrating a method for performing a therapy session to help relieve chronic symptoms. The method is performed by a VE system, similar or identical to the VE system shown in FIG. 1A. The symptoms that can be treated include, for example, hot flashes, anxiety, chemo brain, immune system, stress, pain, fatigue, hair loss, nerve and muscle problems such as numbness and tingling, mood changes, and so on.

At S210, an experience to be trained during the session therapy is selected. In an embodiment, the experience is selected at least based on the goal set of the therapy and personal parameters. In an embodiment, one or more environments may be also considered when selecting the experience. Experiences to can be selected include, but are not limited, to a wintery scenery, a meditation scenery, breathing exercise, experiences utilizing games or gamified environments, and the like. It should be noted that an experience can be rendered using virtual reality techniques, such as, but not limited to 360 VR experience, CGI and the like.

As an example for the operation of S210, if the goal of the session is to relieve hot flashes, the experience to be selected is a wintery scenery. The objects and the exercises to be performed are as determined as part of the experience level.

At S220, the user is asked some questions to determine her current physical conditions. The questions are provided by an AI engine in response to the personal parameters and received answers. Examples for questions are provided above. In an embodiment, the user may choose to skip the initial questions and move straight to the VE.

At S230, in response to the received answer the user determines the initial VE to be rendered and displayed to the user. Following the above example, if the goal of the session is to relieve hot flashes and through the questions it is determined that the frequency of the hot flashes is low, the initial VE would be a cloudy fall day. In contrast, if the frequency the hot flashes is high, the initial VE would be a snowy day.

Furthermore, an initial tolerance may be determined at this stage. The tolerance is the rate of which the cold environment reacts to the hot flash, (i.e. decrease in temperature per second inside the VE). Data gathered from many users can be used to determine the initial tolerance which acts as a default for the VE for all new users.

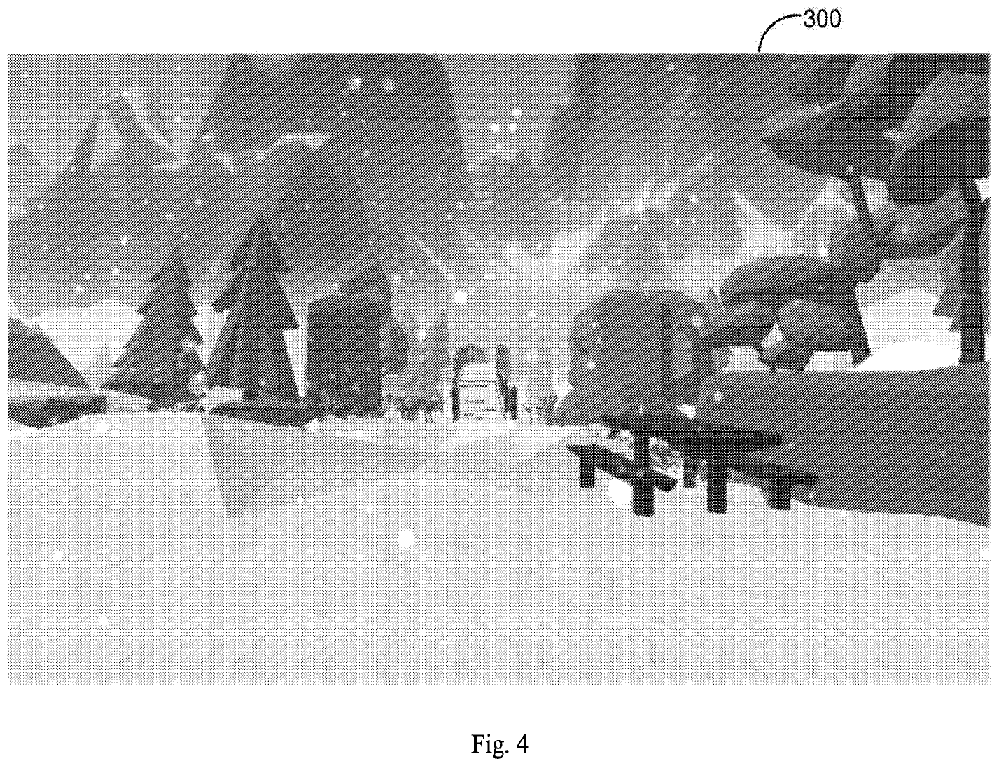

FIG. 4 shows a screenshot of an initial VE 400 demonstrating a snowy day of part of the wintery scenery. This environment is designed to “cool” the user, thus relieve hot flashes. The VE 400 may also include interaction with the environment such as snowflake movement, animal interaction, wind, etc. based on how difficult it is to cool down the user.

Returning to FIG. 2, At S240, sensory signals are collected from one or more biofeedback sensors. At S250, the collected signals are analyzed to determine if the current (initial) VE environment should be modified. As discussed above, the determination may be performed based on deviation from one or more baselines or based on a machine learning classification engine. A modification of the VE is required when the sensory signals indicate that the user does not positively react to the current environment. That is, the therapy session does not meet the goal being set.

If S250 results with a Yes answer, execution continues with S260; otherwise, execution processed to S270. At S260, the VE is modified based on the current analyzed signals. Modification of the VE may include adjusting the visual and/or audio of the VE. At this stage, the tolerance described at S230 can be adjusted to the specific user's needs. Then, at S265, the modified VE is rendered and displayed to the user.

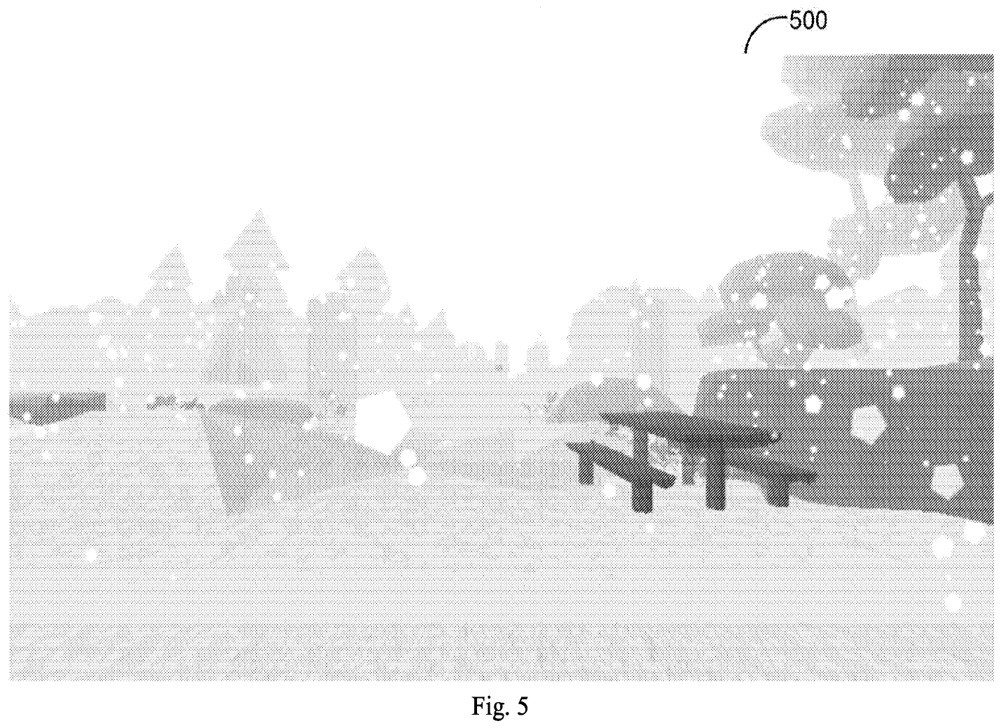

FIG. 5 shows a screenshot of an initial VE 500 demonstrating a severely snowy day of part of the wintery scenery. This environment is a modified version of the environment 500 and utilized when the measured or self-reported body temperature has either increased or stayed the same in response to the VE 400. Alternatively, VE 500 can be utilized initially if the user's initial condition is more severe.

The modified (and also the initial) VE immerses the user in a psychological and therapeutic session. This session helps engage the brain in controlling the physical symptoms and psychological distress during the healing process.

Returning to FIG. 2, At S270, it is checked if the session therapy ends. If so, execution terminates; otherwise, execution returns to S240.

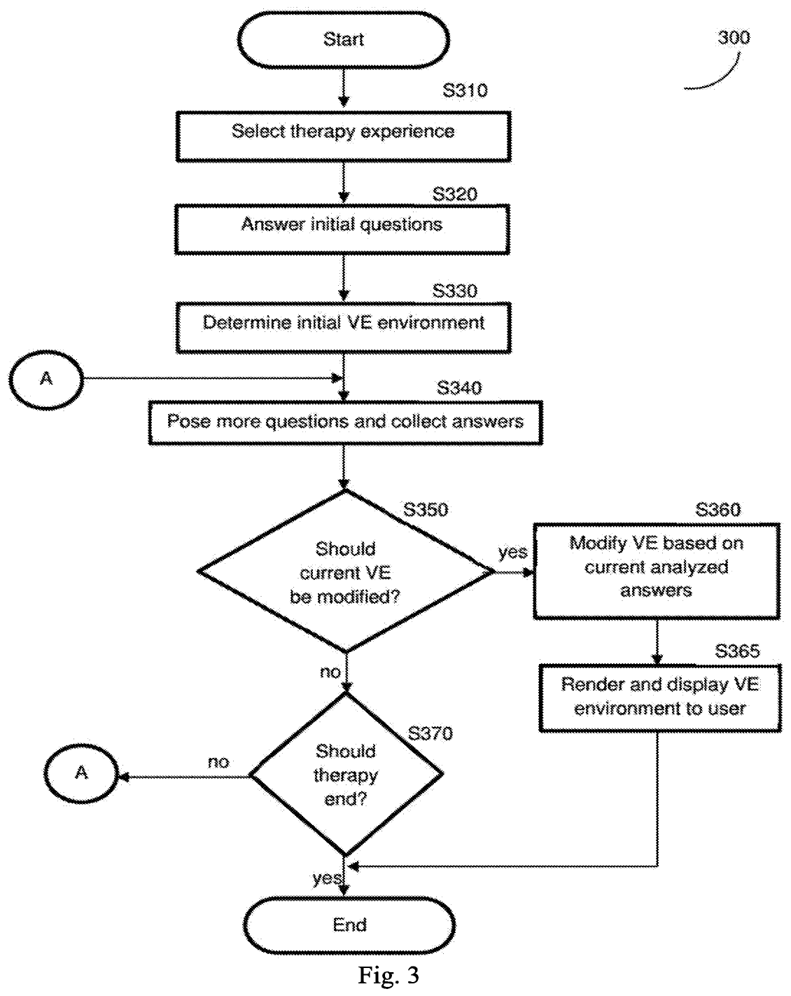

FIG. 3 is an example flowchart 300 illustrating another method for performing a therapy session to help relieve the chronic symptoms. The method is performed by a VE system, similar or identical to the VE system shown in FIG. 1B. The symptoms that can be treated include, for example, hot flashes, anxiety, chemo brain, immune system, stress, pain, fatigue, hair loss, nerve and muscle problems such as numbness and tingling, mood changes, and so on.

At S310, an experience to be trained during the session therapy is selected. In an embodiment, the experience is selected at least based on the goal set of the therapy and personal parameters. In an embodiment, one or more environments may be also considered when selecting the experience. Examples for the personal environments are provided above. Experiences to can be selected in include, but are not limited, to a wintery scenery, a meditation scenery, breathing exercise, experiences utilizing games or gamified environments, and the like. It should be noted that an experience can be rendered using virtual reality techniques, such as, but not limited to 360 VR experience, CGI and the like.

As an example for the operation of S310, if the goal of the session is to relieve hot flashes, the experience to be selected is a wintery scenery. The objects and the exercises to be performed are as determined as part of the experience level.

At S320, the user is asked some questions to determine her current physical conditions. The questions are provided by an AI engine in response to the personal parameters and received answers. Examples for questions are provided above. In an embodiment, the user may choose to go straight to the VE without answering the initial questions.

At S330, in response to the received answer the user determines the initial VE to be rendered and displayed to the user. Following the above example, if the goal of the session is to relieve hot flashes and through the questions is determined that the frequency the hot flashes is low, the initial VE would be a cloudy fall day. In contrast, if the frequency the hot flashes is high, the initial VE would be a snowy day.

At S340, more questions are posed in order to get feedback from the user's experience and answers are collected from the user. Alternatively, the user may choose to experience the VE without answering any more questions. At S350, the collected answers are analyzed to determine if the current (initial) VE should be modified. A modification of the VE is required when the user indicates that she does not positively react to the current environment. That is, the therapy session does not meet the goal being set.

Furthermore, an initial tolerance may be determined at this stage. The tolerance is the rate of which the cold environment reacts to the hot flash based on the user's answers. Data gathered from many users can be used to determine the initial tolerance which acts as a default time for the VE for all new users.

If S350 results with a Yes answer, execution continues with S360; otherwise, execution processed to S370. At S360, the VE is modified based on the current analyzed answers. Modification of the VE may include adjust the visual and/or audio of the VE. At this stage, the tolerance described above can be adjusted to the specific user's needs based on each user answer before, during, and after the experience. Then, at S365, the modified VE is rendered and displayed to the user.

The modified (and also the initial) VE immerses the user in a psychological and therapeutic session. This session helps engage the brain in controlling the physical symptoms and psychological distress during the healing process.

At S370, it is checked if the session therapy ends. If so, execution terminates; otherwise, execution returns to S340.

In some embodiments, during sessions some messages can be displayed to the user with recommendations of the best approach in which to manage the side effects. For example, “accepting the hot flashes as a temporary phenomenon that is part of your recovery . . . ” or “mediating for 15 minutes every day is recommend for the recovery”.

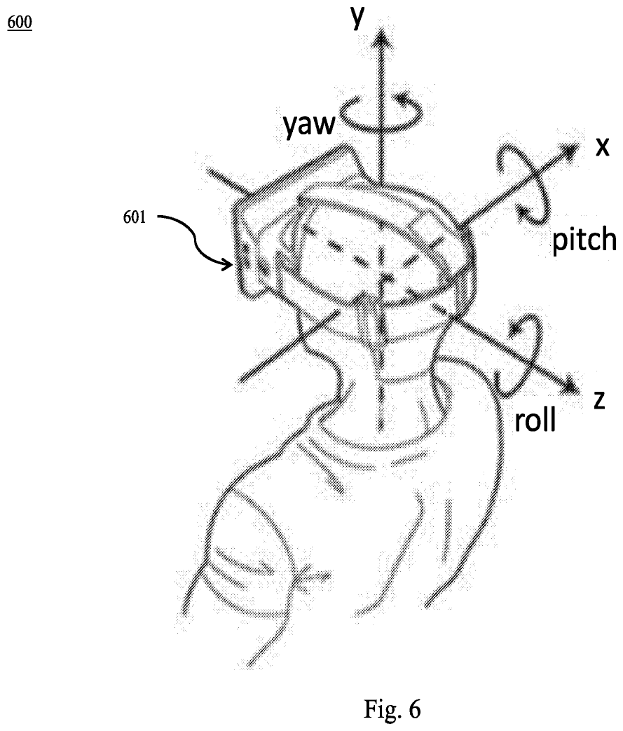

With reference now to FIG. 6, an exemplary virtual reality headset 601 is illustrated according to embodiments of the present disclosure. In various embodiments, system 600 is used to collected data from motion sensors including hand sensors (not pictured), sensors included in headset 601, and additional sensors such as sensors placed on the body (e.g., torso, limbs, etc.) or a stereo camera. In some embodiments, data from these sensors is collected at a rate of up to about 150 Hz. As illustrated, data may be collected in six degrees of freedom: X axis translation—left/right; Y axis translation—up/down/height; Z axis translation—forward/backward; P—pitch; R—roll; Y—yaw. As set out herein, this data may be used to track a user's overall motion to facilitate interaction with a virtual environment and to evaluate their performance. Pitch/Roll/Yaw may be calculated in Euler angles.

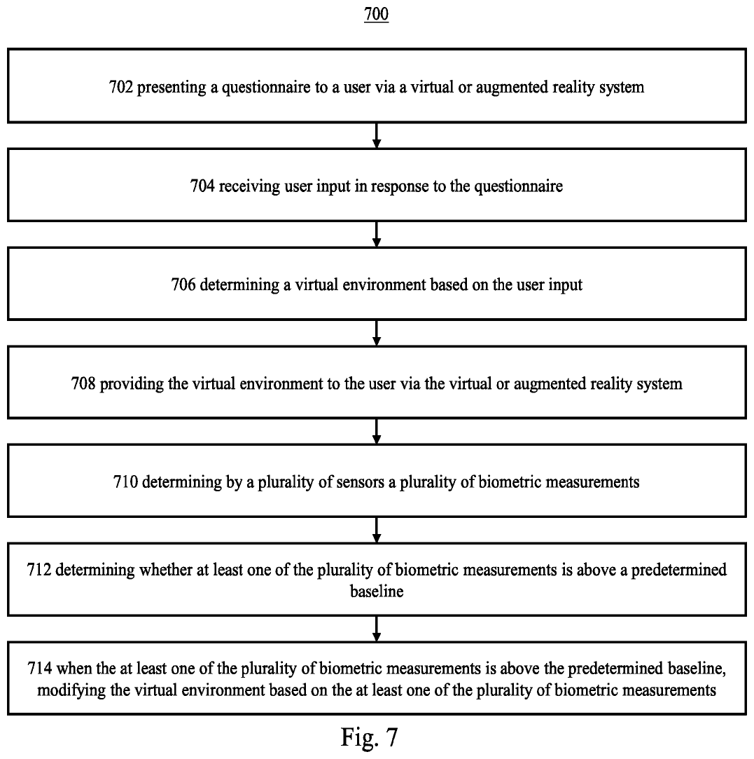

FIG. 7 is a flow chart illustrating an exemplary method 700 for relieving chronic symptoms through treatments in a virtual environment according to embodiments of the present disclosure. At 702, a questionnaire is presented to a user via a virtual or augmented reality system. At 704, user input is received in response to the questionnaire. At 706, a virtual environment is determined based on the user input. At 708, the virtual environment is provided to the user via the virtual or augmented reality system. At 710, a plurality of biometric measurements are determined by a plurality of sensors. At 712, whether at least one of the plurality of biometric measurements is above a predetermined baseline is determined. At 714, when the at least one of the plurality of biometric measurements is above the predetermined baseline, the virtual environment is modified based on the at least one of the plurality of biometric measurements.

In various embodiments, off the shelf VR systems are optionally used with additional external compatible sensors to track various elements in multiple fields including, e.g., motion tracking, cognitive challenges, speech recognition, stability, facial expression recognition, and biofeedback.

Motion tracking can include, but is not limited to tracking of gait, stability, tremors, amplitude of motion, speed of motion, range of motion, and movement analysis (smoothness, rigidity, etc.).

Cognitive challenges can include, but is not limited to reaction time, success rate in cognitive challenges, task fulfillment according to different kind of guidance (verbal, written, illustrated, etc.), understanding instructions, memory challenges, social interaction, and problem solving.

Speech Recognition can include, but is not limited to fluent speech, ability to imitate, and pronunciation.

Stability can include, but is not limited to postural sway.

Bio-Feedback can include, but is not limited to, Heart rate variability (HRV), Electrothermal activity (EDA), Galvanic skin response (GSR), Electroencephalography (EEG), Electromyography (EMG), Eye tracking, Electrooculography (EOG), Patient's range of motion (ROM), Patient's velocity performance, Patient's acceleration performance, and Patient's smoothness performance.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

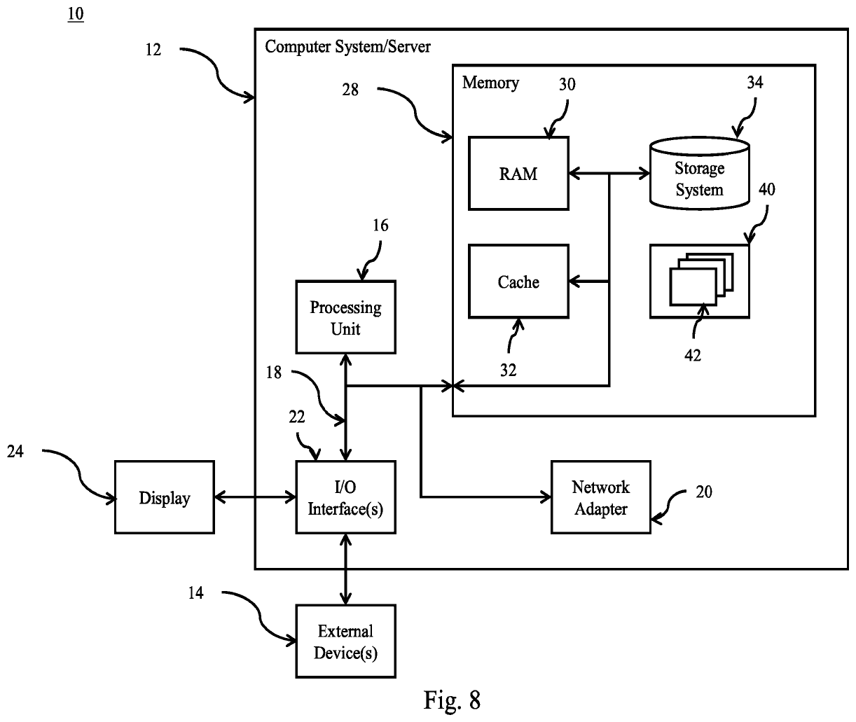

Referring now to FIG. 8, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 8, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

All examples and conditional language recited herein are intended for pedagogical purposes to aid the reader in understanding the principles of the disclosed embodiments and the concepts contributed by the inventor to furthering the art, and are to be construed as being without limitation to such specifically recited examples and conditions. Moreover, all statements herein reciting principles, aspects, and embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method comprising:
presenting a questionnaire to a user via a virtual or augmented reality system;
receiving user input in response to the questionnaire;
determining a virtual environment based on the user input;
providing the virtual environment to the user via the virtual or augmented reality system;
determining by a plurality of sensors a plurality of biometric measurements;
determining whether at least one of the plurality of biometric measurements is above a predetermined baseline; and
when the at least one of the plurality of biometric measurements is above the predetermined baseline, modifying the virtual environment based on the at least one of the plurality of biometric measurements.

2. The method of claim 1, wherein the virtual environment comprises a temperature cue.

3. The method of claim 2, wherein an intensity of the temperature cue is inversely correlated to one of the plurality of biometric measurements of the user.

4. The method of claim 3, wherein the one of the plurality of biometric measurements is body temperature.

5. The method of claim 4, wherein the temperature cue is snow.

6. The method of claim 1, wherein the questionnaire comprises one or more questions regarding a physical condition of the user.

7. The method of claim 6, wherein the one or more questions are related to current mode of feeling, level of intensity and frequency of symptoms or side effects, how the user experienced the symptoms, and/or what was done in the last session to remedy the symptoms.

8. The method of claim 1, wherein determining a virtual environment comprises:
receiving, at a remote server, the user input;
applying a machine learning algorithm to the user input to thereby determine the virtual environment.

9. The method of claim 1, wherein the plurality of biometric measurements comprise body temperature.

10. The method of claim 1, wherein the plurality of biometric measurements comprise breathing rate.

11. The method of claim 1, wherein the plurality of biometric measurements comprise heart rate.

12. The method of claim 1, wherein the plurality of biometric measurements comprise blood oxygen level.

13. The method of claim 1, wherein the plurality of biometric measurements comprise head position, head direction, or head motion.

14. The method of claim 1, wherein the plurality of biometric measurements comprise electrical signals of one or more muscle.

15. The method of claim 1, wherein the plurality of biometric measurements comprise electrical signals of skin.

16. The method of claim 1, wherein the plurality of biometric measurements comprise electrical signals of a brain.

17. The method of claim 1, further comprising:
receiving a therapeutic goal;
receiving one or more parameters of the user;
modifying the virtual environment based on the therapeutic goal and the one or more parameters of the user.

18. The method of claim 17, wherein the therapeutic goal comprises a total number of treatments.

19. The method of claim 17, wherein the therapeutic goal comprises a predetermined value for the one or more of the plurality of biometric measurements.

20. The method of claim 19, wherein the predetermined value comprises a standard value from clinical guidelines.

21. The method of claim 17, wherein the one or more parameters is selected from the group consisting of: age, general medical conditions, personal preferences, disease type, stage of disease, surgical history, drugs being taken, current location, current weather, and current time.

22. The method of claim 1, wherein modifying the virtual environment comprises removing objects from the virtual environment.

23. The method of claim 1, wherein modifying the virtual environment comprises adding objects from the virtual environment.

24. The method of claim 1, wherein modifying the virtual environment comprises modifying visual aspects of the virtual environment to effect a change in one or more of the plurality of biometric measurements for the user.

25. A system comprising:
a virtual or augmented reality display adapted to display a virtual environment to a user;
a plurality of sensors coupled to the user;
a computing node comprising a computer readable storage medium having program instructions embodied therewith, the program instructions executable by a processor of the computing node to cause the processor to perform a method comprising:
presenting a questionnaire to the user via the virtual or augmented reality display;
receiving user input in response to the questionnaire;
determining a virtual environment based on the user input;
providing the virtual environment to the user via the virtual or augmented reality system;
determining, with the plurality of sensors, a plurality of biometric measurements;
determining whether at least one of the plurality of biometric measurements is above a predetermined baseline; and
when the at least one of the plurality of biometric measurements is above the predetermined baseline, modifying the virtual environment based on the at least one of the plurality of biometric measurements.

26. The system of claim 25, wherein the virtual environment comprises a temperature cue.

27. The system of claim 26, wherein an intensity of the temperature cue is inversely correlated to one of the plurality of biometric measurements of the user.

28. The system of claim 27, wherein the one of the plurality of biometric measurements is body temperature.

29. The system of claim 28, wherein the temperature cue is snow.

30. The system of claim 25, wherein the questionnaire comprises one or more questions regarding a physical condition of the user.

31. The system of claim 30, wherein the one or more questions are related to current mode of feeling, level of intensity and frequency of symptoms or side effects, how the user experienced the symptoms, and/or what was done in the last session to remedy the symptoms.

32. The system of claim 25, wherein determining a virtual environment comprises:
receiving, at a remote server, the user input;
applying a machine learning algorithm to the user input to thereby determine the virtual environment.

33. The system of claim 25, wherein the plurality of biometric measurements comprise body temperature.

34. The system of claim 25, wherein the plurality of biometric measurements comprise breathing rate.

35. The system of claim 25, wherein the plurality of biometric measurements comprise heart rate.

36. The system of claim 25, wherein the plurality of biometric measurements comprise blood oxygen level.

37. The system of claim 25, wherein the plurality of biometric measurements comprise head position, head direction, or head motion.

38. The system of claim 25, wherein the plurality of biometric measurements comprise electrical signals of one or more muscle.

39. The system of claim 25, wherein the plurality of biometric measurements comprise electrical signals of skin.

40. The system of claim 25, wherein the plurality of biometric measurements comprise electrical signals of a brain.

41. The system of claim 25, further comprising:
receiving a therapeutic goal;
receiving one or more parameters of the user;
modifying the virtual environment based on the therapeutic goal and the one or more parameters of the user.

42. The system of claim 41, wherein the therapeutic goal comprises a total number of treatments.

43. The system of claim 41, wherein the therapeutic goal comprises a predetermined value for the one or more of the plurality of biometric measurements.

44. The system of claim 43, wherein the predetermined value comprises a standard value from clinical guidelines.

45. The system of claim 41, wherein the one or more parameters is selected from the group consisting of: age, general medical conditions, personal preferences, disease type, stage of disease, surgical history, drugs being taken, current location, current weather, and current time.

46. The system of claim 25, wherein modifying the virtual environment comprises removing objects from the virtual environment.

47. The system of claim 25, wherein modifying the virtual environment comprises adding objects from the virtual environment.

48. The system of claim 25, wherein modifying the virtual environment comprises modifying visual aspects of the virtual environment to effect a change in one or more of the plurality of biometric measurements for the user.

49. A computer program product for relieving chronic conditions in a user comprising a computer readable storage medium having program instructions embodied therewith, the program instructions executable by a processor to cause the processor to perform a method comprising:
presenting a questionnaire to a user via a virtual or augmented reality system;
receiving user input in response to the questionnaire;
determining a virtual environment based on the user input;
providing the virtual environment to the user via the virtual or augmented reality system;
determining by a plurality of sensors a plurality of biometric measurements;
determining whether at least one of the plurality of biometric measurements is above a predetermined baseline; and
when the at least one of the plurality of biometric measurements is above the predetermined baseline, modifying the virtual environment based on the at least one of the plurality of biometric measurements.

50. The computer program product of claim 49, wherein the virtual environment comprises a temperature cue.

51. The computer program product of claim 50, wherein an intensity of the temperature cue is inversely correlated to one of the plurality of biometric measurements of the user.

52. The computer program product of claim 51, wherein the one of the plurality of biometric measurements is body temperature.

53. The computer program product of claim 52, wherein the temperature cue is snow.

54. The computer program product of claim 49, wherein the questionnaire comprises one or more questions regarding a physical condition of the user.

55. The computer program product of claim 54, wherein the one or more questions are related to current mode of feeling, level of intensity and frequency of symptoms or side effects, how the user experienced the symptoms, and/or what was done in the last session to remedy the symptoms.

56. The computer program product of claim 49, wherein determining a virtual environment comprises:
receiving, at a remote server, the user input;
applying a machine learning algorithm to the user input to thereby determine the virtual environment.

57. The computer program product of claim 49, wherein the plurality of biometric measurements comprise body temperature.

58. The computer program product of claim 49, wherein the plurality of biometric measurements comprise breathing rate.

59. The computer program product of claim 49, wherein the plurality of biometric measurements comprise heart rate.

60. The computer program product of claim 49, wherein the plurality of biometric measurements comprise blood oxygen level.

61. The computer program product of claim 49, wherein the plurality of biometric measurements comprise head position, head direction, or head motion.

62. The computer program product of claim 49, wherein the plurality of biometric measurements comprise electrical signals of one or more muscle.

63. The computer program product of claim 49, wherein the plurality of biometric measurements comprise electrical signals of skin.

64. The computer program product of claim 49, wherein the plurality of biometric measurements comprise electrical signals of a brain.

65. The computer program product of claim 49, further comprising:
receiving a therapeutic goal;
receiving one or more parameters of the user;
modifying the virtual environment based on the therapeutic goal and the one or more parameters of the user.

66. The computer program product of claim 65, wherein the therapeutic goal comprises a total number of treatments.

67. The computer program product of claim 65, wherein the therapeutic goal comprises a predetermined value for the one or more of the plurality of biometric measurements.

68. The computer program product of claim 67, wherein the predetermined value comprises a standard value from clinical guidelines.

69. The computer program product of claim 65, wherein the one or more parameters is selected from the group consisting of: age, general medical conditions, personal preferences, disease type, stage of disease, surgical history, drugs being taken, current location, current weather, and current time.

70. The computer program product of claim 49, wherein modifying the virtual environment comprises removing objects from the virtual environment.

71. The computer program product of claim 49, wherein modifying the virtual environment comprises adding objects from the virtual environment.

72. The computer program product of claim 49, wherein modifying the virtual environment comprises modifying visual aspects of the virtual environment to effect a change in one or more of the plurality of biometric measurements for the user.
